(19) United States
(12) Patent Application Publication
(10) Pub. No.: US 2023/0225613 A1
(43) Pub. Date: Jul. 20, 2023

(54) METHOD AND A SYSTEM FOR A NON-INVASIVE ASSESSMENT OF A RELATION BETWEEN AN INTRACRANIAL PRESSURE AND AN INTRAOCULAR PRESSURE

(71) Applicant: STATUMANU ICP APS, Glostrup (DK)

(72) Inventor: Jakob Find Munk Madsen, Glostrup (DK)

(21) Appl. No.: 18/009,683

(22) PCT Filed: Jun. 17, 2020
(86) PCT No.: PCT/DK2020/050174

§ 371 Date: Dec. 9, 2022

(51) Int. Cl. A61B 3/16; A61B 5/0205; A61B 3/14; A61B 3/12; A61B 3/00; A61B 5/03; A61B 5/00

(52) U.S. Cl. CPC A61B 3/16; A61B 5/0205; A61B 3/14; A61B 3/12; A61B 3/0025; A61B 5/031; A61B 5/489; A61B 5/024

(57) ABSTRACT

Method and system for a non-invasive assessment of a relation between an intracranial pressure and an intraocular pressure. The method comprising the steps of recording a plurality of images of a retina part of an eye of a person, identifying at least one vein, determining a first plurality of characteristic vein diameters for the identified vein at a first vein location, determining whether the at least one vein has experienced a vein collapse during the first time period, and determining a relation between intraocular pressure and intracranial pressure during the first time period.

### CROSS-REFERENCE

The present application is the national phase entry under 35 U.S.C. 371 of International Patent Application No. PCT/DK2020/050174 by Madsen, entitled “METHOD AND A SYSTEM FOR A NON-INVASIVE ASSESSMENT OF A RELATION BETWEEN AN INTRACRANIAL PRESSURE AND AN INTRAOCULAR PRESSURE”, filed Jun. 17, 2020, which is assigned to the assignee hereof and is incorporated by reference in its entirety.

### TECHNICAL FIELD

The present disclosure relates to a method and a system for a non-invasive assessment of a relation between intracranial pressure (ICP) and intraocular pressure (IOP).

### BACKGROUND

The measurement of ICP is an important tool in connection with diagnosing different health disorders, such as head injuries, stroke oedema, intracranial haemorrhage, as an overpressure is potentially fatal. Traditionally, the ICP has been determined by drilling a hole in the skull and inserting a manometer. Needless to say, that such an invasive method is potentially dangerous, not only as such, but also indirectly due to risk of infection. Accordingly, various prior art methods for non-invasive measurement of ICP have been proposed, some of which rely on inspection of the optical arteries which supply the eyes with blood. These arteries run from inside the skull to the eyes and are thus influenced by the pressure within the skull. One type of non-invasive method is disclosed in WO 2016/11637. WO 2016/11637 is a prior application by the applicant, which discloses a method for non-invasive assessment of ICP. The prior art method comprises the steps of recording at least one image of an eye of a person, identifying at least one artery and at least one vein, calculating an arteriovenous ratio (AVR), and comparing said AVR with a threshold value to estimate intercranial pressure. Such a method is both fast, efficient and puts minimal stress on a person undergoing the non-invasive assessment.

The prior art method relies on the acknowledgement that the walls of veins and arteries are of different strength. Veins generally exhibit a lower strength than arteries and are therefore more prone to deformation caused an increase or a decrease in vein pressure. Therefore, a high pressure within the vein will lead to the vein expanding more than the artery, thus lowering the AVR, which may indicate an elevated ICP.

Furthermore, it has long been known that the retinal veins pulsate and sometimes even collapse. The pulsation of veins has been correlated with ICP. The correlation between pulsation of veins and ICP is further explained by William H. Morgan, Christopher R. P. Lind, Samuel Kain, Naeem Fatehee, Arul Bala, Dao-Yi Yu, “Retinal Vein Pulsation Is in Phase with Intracranial Pressure and Not Intraocular Pressure”, Investigative Ophthalmology & Visual Science, 2012; 53(8):4676-4681, doi:10.1167/iovs.12-9837, which showed the retinal vein pulsation is in phase with ICP and not IOP.

Older studies have also documented how the presence of vein pulsation may be a reliable indicator of ICP below 180 to 190 mmH₂O (13.2 mmHg to 14 mmHg), cf. Barry E. Levin, “The Clinical Significance of Spontaneous Pulsations of the Retinal Vein”, Archives of neurology, 1978; 35(1):37-40, doi:10.1001/archneur.1978.00500250041009.

Newer studies describe, how vein pulsations are in fact caused by variation in the pressure gradient along the retinal vein as it traverses the lamina cribrosa, cf. A. S. Jacks, N. R. Miller “Spontaneous retinal venous pulsation: aetiology and significance”, Journal of Neurology, Neurosurgery & Psychiatry, 2003; 74:7-9, doi:10.1136/jnnp.74.1.7. The pressure gradient varies because of the difference in the pulse pressure between the intraocular space and the cerebrospinal fluid. The importance of this is that as the ICP rises the intracranial pulse pressure rises to equal the intraocular pulse pressure and the spontaneous venous pulsations cease. Thus, the cessation of the spontaneous venous pulsation is a sensitive marker of raised ICP.

Even though it has been established that a relation between ICP and IOP is present and it affects vein pulsation, the use of this information has still not been implemented in a meaningful manner. A reason for this may be that, so far, a reliable method for assessing the relation between ICP and IOP, in a simple, fast and efficient manner have not been developed.

### SUMMARY

Based on this prior art it is an object according to a first aspect of the present disclosure to provide a non-invasive method for assessing a relation between an ICP and an IOP in a simple, fast and efficient manner.

According to a first aspect of the present disclosure, this object is achieved by a method for a non-invasive assessment of a relation between an ICP and an IOP using an image recording device, said method comprising the steps of:
a) recording, over a first time period, a plurality of images of a retina part of an eye of a person using said image recording device,
b) identifying, in a first plurality of images, at least one vein,
c) determining, in the first plurality of images from the plurality of images recorded over the first time period, a first plurality of characteristic vein diameters for the identified vein at a first vein location,
d) determining, based on the first plurality of characteristic vein diameters, whether the at least one vein has experienced a vein collapse during the first time period, and
e) determining a relation between IOP and ICP during the first time period, wherein if the at least one vein has experienced a vein collapse the IOP is determined to exceed the ICP.

Being able to determine the relation between IOP and ICP by simply recording images of an image and determining whether a vein has collapsed allows for a simple, fast and efficient method for assessing the relation between the ICP and the IOP. This, in return, may assist in determining whether a person has an elevated ICP. Furthermore, methods relying on measuring AVR for the determination of ICP, such as presented by the applicant's earlier application WO 2016/11637 A1 which is hereby incorporated by reference, may be improved upon as vein pulsation may result in large changes in the AVR during just one heart pulse cycle. Thus, being able to determine whether a vein collapse has happened allows one to take this into account when measuring the AVR.

In the context of the disclosure a non-invasive assessment is to be understood as an assessment not requiring any surgical procedure, requiring minimal to no physical contact to a person undergoing the assessment, and not harming the person.

In the context of the disclosure IOP is to be understood as the fluid pressure inside the eye. IOP is as a standard measured in mmHg, a normal IOP is between 12 mmHg and 22 mmHg, though several conditions may lead to ocular hypertension resulting in IOPs above 22 mmHg, and other conditions may lead to ocular hypotension resulting in IOPs below 12 mmHg.

In the context of the disclosure ICP is to be understood as the fluid pressure inside the skull. ICP is as a standard measured in mmHg, a normal ICP is between 7 mmHg and 15 mmHg, though several conditions may lead to intracranial hypertension resulting in ICPs above 15 mmHg, and other conditions may lead to intracranial hypotension resulting in ICPs below 7 mmHg.

In the context of the disclosure the image recording device may be any suitable device. This could be a dedicated device for this specific purpose. It could also be a digital camera with suitable optics, preferably in combination with a processing unit, such as a personal computer, PC, for inter alia processing the image data according to the method, and possibly providing storage capacity for the recorded images, at least temporarily. In particular, however, the image recording device could be the built-in camera of a smart phone fitted with a suitable lens adapter. The smart phone could thus be used both for the recording of the images, and the subsequent image data processing according to the method, as well as providing storage capacity for the recorded images. Suitable lens adapters for recording eye images are commercially available, such as the iExaminer™, from Welch Allyn, Inc., 4341 State Road, Skaneateles Falls, N.Y. 13153, USA.

According to the method a plurality of images of a retina part of an eye of a person is recorded. The plurality of images may be constituted by two images of the retina part of the image. The plurality of images may be constituted by more than two images of the retina part, preferably the number of images in the plurality of images should be sufficient for determining an average or median AVR over the first time period. The number of images in the plurality of images may depend on the capture rate of the image recording device and the length of the first time period. Preferably, the capture rate of the image recording device may be determined to at least satisfy the Nyquist rate. By satisfying the Nyquist rate alias free signal sampling may be achieved. The capture rate may for example as a minimum be 3-6 frames per second (fps), though using modern cameras for recording allows for capture rates of 3-60 fps. The capture rate may be chosen depending on the exposure time required to take images of sufficient quality to be used in the method according to the disclosure.

The first time period preferably corresponds to at least one cardiac cycle, also referred to in this application as a heart pulse cycle. Having the first time period at least corresponding to one heart pulse cycle assures that images may record an image of a vein at both a maximum and minimum pressure within the vein. In some examples the first time period may also correspond to two heart pulse cycles, three heart pulse cycles, four heart pulses cycle, five heart pulse cycles, or more. Recording images over a longer period may help in giving more usable images, thus leading to an improvement in determining whether the vein has collapsed during the recorded time period. Furthermore, if the recorded images are to be used for determining the AVR, recording over a longer time may result in a better determination of the AVR. However, under some circumstances it may be preferable to keep the recording time short, e.g.

in emergency situations where a fast measurement may be preferable. The time period over which images are recorded may preferably be 20-60 seconds, 10-120 seconds, or 5-180 seconds.

The at least one vein may be identified by a person visually inspecting the plurality of images recorded, but this is preferably performed in an automated process by image processing software, running on a dedicated device, on an associated processing unit, or on a smart phone.

The at least one vein may be identified using an edge filtering method that leaves an edge filtered image showing edges only. This is one of the reasons why compressed images are unwanted, as the desire is to have sharp edges of transitions in the images, and not artificially blurred edges by compression. For the same reason any automatic filtering and edge enhancement in the camera should preferably be disposed of, suppressed or otherwise avoided. This is in particular the case if the camera is the built-in camera of a smart phone, where such features are commonplace.

To filter whether a recorded image is usable, a mean filtering may be applied to the edge filtered image. In the mean filtering, the edge filtered image is broken down in blocks of e.g. 10×10 pixels, 25×25 pixels 50×50 pixels, or 100×100 pixels. In these blocks the image processing software determines the frequency of edges within each of the blocks. The image can then be classified according to the distribution between blocks having a low frequency of edges and blocks having a high frequency of edges. If a recorded image is blurred the edge filtered images will yield very few or no blocks with a high frequency of edges, and can therefore, be rejected or accepted by the image processing software, based on a pre-set threshold.

Having determined that the quality of the recorded image is appropriate, the vessels are identified among the other features in the recorded image. This identification of vessels may also be done by a person visually inspecting the image. However, preferably the identification is performed in an automated process by the image processing software, running on the dedicated device, on the associated processing unit, or on the smart phone.

The identification of the vessels among the other features may be performed in various ways, based on known image analysis methods, or by visual inspection of an image by a person. A person will normally not have problems identifying blood vessels in the recorded images. Areas identified by the person as blood vessels, may simply be marked by mouse click or similar, when the person views the recorded image on a display. However, since a fully automated method that can be implemented in a device is desired, Gauss line analysis may be used. Gauss line analysis is well known, and implemented in existing image processing software, such as Halcon 12 from MVTec Software GmbH.

The Gauss line analysis can be fully automated and performed by imaging processing software to find lines in images. It should be noted that the term lines is not to be understand in a narrow mathematical sense as one-dimensional straight lines but is to be understand as lines with a certain width, as well as curves and other features. The width of the lines corresponds to the characteristic diameter of the blood vessel, i.e. vein or artery. Because there are both arteries and veins and both of these types of blood vessels are branched, line segments with more or less constant widths rather than continuous lines will be identified. The Gauss line analysis yields the width at each point along all these line segments. The Gauss line analysis, however, will not discriminate between arteries and veins, and this will have to be performed in a separate step.

As a first alternative to Gauss line analysis, a texture analysis with subsequent discriminant analysis, as described in WO 2006/042543, could be used to identify major and minor blood vessels and other features, such as fundus, the optic disc, out of image areas, edges between e.g. optic disc and fundus, etc. It is thus to be understood that an identification of the optic disc, need not be performed as a separate subsequent step of the method according to the disclosure. In addition to texture, other parameters, mean values of colours (R, G, B), and variance of colours (R, G, B) may be used to convert the image data to classes. This texture analysis itself will, however, not yield any discrimination between arteries and veins, and this will still have to be performed in a separate step.

A second alternative would be manual selection by a person visually inspecting the image on a screen, and marking blood vessels using a suitable marker known per se, such as a computer mouse and pointer, a stylus on a touch screen or the like.

More alternatives exist and in fact the libraries of the Halcon 12 software comprise a number of preprogramed software algorithms for blood vessel detection.

The efficiency and reliability of the various image processing methods for finding the blood vessels in an image may depend on the actual image, or the quality thereof. That is to say, one method may provide a reliable result when used on one image, and a less reliable result, such as an ambiguous result, or even fail entirely, when used on another image. It may therefore be desired to subject each image to several of the available image analysis methods, and combine the results for increased reliability.

In the process of identifying the vessels an experienced person would at the same time be able to discriminate between veins and arteries, and identify pairs of corresponding arteries and veins. In doing so there are a few general rules that are helpful, not only in the visual inspection, but also in any automated process. The largest pair of veins and arteries in the image would in most persons extend in a generally vertical direction upward from the optic disc where veins arteries enter the eye along the optical nerve and second largest pair would extend downward from the centre of the optic disc. This a general rule and there are individual differences between persons and exceptions. In respective pairs of veins and arteries the veins will generally have a larger diameter than the arteries and thus be wider in an image. Both arteries and veins branch out in a somewhat fan shaped manner, meaning the artery vessels do not cross each other in the image, and vein vessels do not cross each other in the image. Thus, if vessels cross, one must be an artery and the other a vein or vice versa. As for the major veins and arteries extending in the vertical direction upwardly and downwardly from the centre of the optic disc above and below the centre of the optic disc, the will generally be relatively close to each other over at least one segment and readily identifiable as a pair.

For an automated system, however, it is convenient to start with the fact that a vein generally has a larger diameter than its corresponding artery. Accordingly, diameters of vessels are determined at one or more distances from the centre of the optic disc (or alternatively from a point at the optic nerve at which veins and arteries generally converge.

The method is not limited to just one vein, but may also be carried on a plurality of veins. In some examples a plurality of veins is identified, and the plurality of identified veins is used for identifying whether one or more veins has collapsed. In some examples where a plurality of veins has collapsed the method may also output the number of veins from the plurality of vein, which has experienced a vein collapse. By identifying a plurality of veins, a more reliable measurement may be made.

The first plurality of images may be comprised by two or more images from the plurality of recorded images. The first plurality of images being a plurality of images for the determination of a characteristic vein diameter at a first vein location, and preferably also suitable for a determination of a characteristic artery diameter at a first artery location. The first plurality of images comprising at least two images taken at different time windows within the first time period. According to some examples it may be sufficient to have the first plurality of images consisting of an image of the vein during systole and an image of the vein during diastole.

The determination of the first plurality of characteristic vein diameters may be carried out manually by a person. A manual determination may for example be carried out by a person marking two different points on at least two different images from the first plurality of imagen, and then having a dedicated device, associated processing unit, or a personal computer returning the distance between the marked points. Preferably the determination of the plurality of characteristic vein diameters is carried out automatically by appropriate image software, running on a dedicated device, associated processing unit, or a personal computer. The plurality of characteristic vein diameters may be average diameters of the vein over a distance of the vein, or widths of the vein determined between two points on the vein.

A characteristic vein/artery diameter in the context of the disclosure is to be understood as a width of the vein/artery as seen in the imaged plane. Even though the wording diameter is used, it should not be interpreted narrowly as only being applicable to circles or ellipses. In the context of the disclosure diameter is to be understood as a width of the vein/artery in the imaged plane, even though the arteries and/or veins assume cross-section which are not circular or elliptical.

The first vein location may be any location on the retina of the imaged eye, which comprises a vein.

A vein collapse in the context of the disclosure is to be understood as a vein undergoing a flattening of the cross-section, e.g. going from having a substantially circular cross-section to having a substantially elliptical cross-section or otherwise flattened cross-section. Blood vessels in the body may of course may assume cross-sections differing from a circular cross-section the mention of circular cross-sections and elliptical cross-sections in this disclosure is merely meant for ease of understanding, however this disclosure is not only limited to these cross-sections, A vein collapse in the context of the disclosure is any vein undergoing a flattening of the cross-section.

The vein collapse will generally happen in the imaged plane, thus when a collapsed vein is imaged it is generally imaged along the semi major axis of the elliptical cross-section. Thus, when the vein experiences a collapse the characteristic vein diameter will undergo an increase, as a result of the eccentricity of the elliptical cross-section. A vein collapse is caused by the IOP exceeding the ICP.

The determination on whether the at least one vein has experienced a vein collapse during the first time period may be carried out in a plethora of ways. A simple way of determining whether a vein has collapsed may be to track the change in characteristic vein diameter over the first time period, e.g. to track whether the characteristic vein diameter increases from going the systolic blood pressure to diastolic blood pressure. Other ways of determining whether a vein has experienced a vein collapse during a first time period will be presented later on in this application.

Determining the relation between IOP and ICP during the first time period, need not be performed as an explicit additional step, but may instead be considered as an implicit step performed as a consequence of the determination whether the vein has undergone a collapse.

The relation between IOP and ICP may alternatively be explicitly outputted as part of the method. The output may be a message on a display associated with the image recording device.

In an example the step d) further comprises:
d.1) identifying, in said plurality of images, at least one artery associated with said vein,
d.2) determining, in the first plurality of images from the plurality of images recorded over the first time period, a first plurality of characteristic artery diameters for the identified artery at a first artery location,
d.3) determining, based on the first plurality of characteristic artery diameters, an artery diameter behaviour,
d.4) determining, based on the first plurality of characteristic vein diameters, a vein diameter behaviour,
d.5) comparing the vein diameter behaviour to the artery diameter behaviour, and
d.6) determining, based on the comparison between the vein diameter behaviour and the artery diameter behaviour, whether the at least one vein has experienced a vein collapse during the first time period.

As the strength of the walls of arteries are several times higher than that of veins, arteries do not experience collapses as seen in veins. Though, uniform changes in the size of the artery's cross-section is still observed at different pressures. The change in artery cross-section is a uniform expansion when the pressure increases within the artery, and a uniform contraction when the pressure decreases within the artery. The uniform increase or decrease of the artery cross-section gives a corresponding increase or decrease in the characteristic artery diameter. In the situation where the vein does not collapse, the general behaviour of the vein will match that of the artery, i.e. increases and decreases in pressure within the vein will lead to corresponding increases or decreases in the characteristic vein diameter. However, when the vein undergoes a collapse the behaviour between the vein and artery differs. The vein collapses when the ICP decreases and reaches a value below the IOP. The collapse of the vein leads to an increase in the characteristic vein diameter. The cause for the increase in the characteristic vein diameter is that the vein collapses in the imaging plane, thus the eccentricity of the elliptical shape caused by the vein collapse leads to an increased characteristic vein diameter. Therefore, in the situation where the vein collapses the vein will exhibit a different behaviour from the artery, since the artery will experience a decrease in the characteristic diameter while the vein will experience an increase in the characteristic diameter. Thus, by comparing the vein diameter behaviour to the artery diameter behaviour to see if both exhibit similar behaviour or differing behaviour during the first time period it is possible to determine, whether the vein has collapsed during the first time period.

An artery diameter behaviour and a vein diameter behaviour are in the context of the disclosure to be understood as a behaviour of the characteristic artery diameter and a behaviour of the characteristic vein diameter over a given time period. The behaviour may be understood as the absolute or relative changes in characteristic artery diameter and characteristic vein diameter over a given time period.

The at least one artery associated with the at least one vein may be identified may be identified and analysed in a corresponding manner as the at least one vein.

In an example the plurality of images of the retina part of the eye are also of an optic disc of the eye, wherein the step d) further comprises:
d.7) determining, in the first plurality of images from the plurality of images recorded over the first time period, the location of the optic disc,
d.8) determining, in the first plurality of images from the plurality of images recorded over the first time period, a second plurality of characteristic vein diameters for the identified vein at a second vein location, wherein the second vein location is farther away from the optic disc than the first vein location,
d.9) determining, based on the first plurality of characteristic vein diameters, a first vein diameter behaviour,
d.10) determining, based on the second plurality of characteristic vein diameters, a second vein diameter behaviour,
d.11) comparing the first vein diameter behaviour to the second vein diameter behaviour, and
d.12) determining based on the comparison between the first vein diameter behaviour and the second vein diameter behaviour, whether the at least one vein has experienced a vein collapse during the first time period.

From observation it has been observed that when the vein collapses it happens close to the optic disc, where the vein exits the eye. The collapse of the vein close to the optic disc leads to an increased pressure within the vein upstream from the collapse. The increased pressure upstream from the collapse prevents the collapse from propagating further upstream the vein, thus leading to the collapse only being a localized incident on the vein. Thus, by observing the characteristic vein diameter behaviour at a first vein location and then comparing it to the characteristic vein diameter behaviour at a second vein location it is possible to determine whether a vein collapse has occurred. Since if the behaviour between the two locations differs it indicates a vein collapse has happened.

The optic disc may be identified in a similar manner as the veins and/or arteries. The optic disc is quite easily distinguishable, because it is much brighter than the fundus as such. Because the optic disc is much brighter than the rest of the fundus, identifying it is also quite easily carried out in an automated process using image processing software. The optic disc, or at least a representative location thereof, may be identified based on the vessels, which all enter the eye along the optical nerve at the centre of the optic disc. The optic disc may be found by shape search by the image processing software. The optic disc may be found by image correlation where the image processing software searches for the best correlation in the image with an image of a circular disc. As with the identification of the veins and/or arteries, several different image processing methods could be used on each image and a combined result be used.

Although only a first vein location and a second vein location has been mentioned the disclosure is not limited to this, a third vein location, a fourth vein location, etc. may also be used. Alternatively, the characteristic diameter of the vein may be determined continuously over a part of the vein.

In an example the second vein location is at least a distance corresponding to a diameter of the optic disc away from the optic disc.

In the field of ophthalmology, the use of absolute terms is seldom used, since the anatomy of people differs. Thus, relative terms are more commonly, such as the diameter of the optic disc. Having the second vein location at least a distance corresponding to a diameter of the optic disc away from the optic disc, it assures that the second vein location is far enough away from the optic disc to not undergo a collapse, thus assuring the collapse, if present, is only present at the first vein location.

In an example the step d) further comprises:
d.13) determining, based on the first plurality of characteristic vein diameters, a change in vein diameter during the first time period,
d.14) comparing the change in vein diameter with a threshold value, and
d.15) determining, based on the comparison between the change in vein diameter and the threshold value, whether the at least one vein has experienced a vein collapse during the first time period.

The change in vein diameter happening as a result of the vein collapsing, has been observed by the applicant to significantly exceed the change observed when the vein undergoes uniform expansion and uniform contraction caused by changing pressures during a cardiac cycle. Thus, comparing the change in vein diameter during the first time period to a threshold, allows for the detection of whether the vein has collapsed during the first time period or has not collapsed during the first time period.

The relative change in the characteristic vein diameter when the vein undergoes uniform expansion and uniform contraction is generally below 2%, where the relative change in the characteristic vein diameter when the vein undergoes a collapse is above 2% and may even reach 5-6% or higher. Thus, the threshold may be set at a relative change in the characteristic vein diameter of 2%, 3%, 4%, 5%, or 6%.

The change in vein diameter determined is preferably determined as the maximum change in vein diameter during the first time period.

In an example the method further comprises,
f) repeating the steps, a-e), wherein the step a) is repeated for a second time period.

Repeating the steps of the method for a second time period allows for determining the relation between the IOP and the ICP for the second time period. Having the relation between the IOP and the ICP for two different time periods allow for determining the development between the IOP and the ICP, which in return may indicate whether the ICP is rising or decreasing.

The second time period may be substantially equal to first time period in length or it may differ from the first time period in length. The first time period and the second time period may be time periods separated by a considerable amount of time, e.g. one day, two days, three days, one week, two week, three weeks, one month, two months, three months, even longer, or any duration between the mentioned times. Having the time periods a considerable amount of time apart may allow for monitoring a person over a prolonged time window. The first time period and the second time period may be time periods separated by a shorter amount of time, e.g. one minute, two minute, fifteen minutes, thirty minutes, an hour, two hours, three hours, or any duration between the mentioned times. Having the time periods close to each other may allow for monitoring of a person in an emergency situation, where the situation can develop rapidly.

The steps of the method are not limited to only being repeated for a second time period but may further be repeated for a third time period, a fourth time period, a fifth time period, or further time periods.

In an example the method further comprises,
g) identifying, in said plurality of images, at least one artery associated with said vein,
h) determining, in the first plurality of images from the plurality of images recorded over the first time period, a first plurality of characteristic artery diameters for the identified artery at a first artery location,
i) calculating an AVR based on the first plurality of characteristic artery diameters and the first plurality of characteristic vein diameters, and
j) comparing the AVR to the relation between IOP and ICP during the first time period.

Determining both the relation between IOP and ICP and the AVR may provide enough information to determine, whether a person has an elevated cranial pressure. Furthermore, as the AVR is intrinsically linked to the relation between IOP and ICP, the determined AVR may be interpreted in view of the determined relation. The calculated AVR may also be compared with a threshold value. The comparison of the AVR with a threshold may allow for a non-invasive assessment of the ICP of a person. The threshold may be set in part based on the relation between the ICP and the IOP. The previous application by the applicant WO 2016/11637 A1 describes how the AVR and the threshold may be used for assessing the ICP.

The calculated AVR may be calculated as an average AVR over the recorded time period. The calculated AVR may be calculated as a median AVR over the recorded time period. In an example the method further comprises,
k) repeating the steps g-i), wherein the steps g-i) is repeated for the second time period,
l) determining the change in AVR between the first time period and the second time period, and
m) comparing the change in AVR to the relation between IOP and ICP during the first time period and to the relation between IOP and ICP during the second time period.

Having both the change in AVR and the relation between IOP and ICP for two different time periods, allows for a precise assessment on the development of the ICP between time periods.

In an example the first time period and/or the second time period is at least equal to a duration of one heart pulse cycle of the person and/or at least one respiratory cycle for the person.

Recording over a duration of one heart pulse cycle of the person and/or at least one respiratory cycle for the person allows for recording the vein, when the pressure inside the vein is at a maximum and when the pressure inside the vein is at a minimum. Thus, assuring that if the vein undergoes a collapse it is recorded.

The heart pulse cycle of the person and/or at least one respiratory cycle for the person may be measured by an external device, such as an electrocardiograph machine, a pulse oximeter, or a heart rate sensor.

In an example the method further comprises:
determining, based on the first plurality of characteristic vein diameters, the duration of at least one heart pulse cycle of the person and/or at least one respiratory cycle of the person.

Consequently, the need for external devices for determining the respiratory and/or the heart pulse cycle is made obsolete, since it is possible to directly determine the duration from the recorded images. The determination may be made by tracking the change in the first plurality of characteristic vein diameters over the recorded time period.

In a second aspect of the disclosure it is an object to provide a system for performing a non-invasive assessment of a relation between an ICP and an IOP, said system comprising
an image recording device, configured to record, over a first time period, a plurality of images of a retina part of an eye of a person,
a processing unit communicatively connectable to the image recording device and configured to:
receive the plurality of images recorded by the image recording device,
identify, in said plurality of images, at least one vein,
determine, in a first plurality of images from the plurality of images recorded over the first time period, a first plurality of characteristic vein diameters for the identified vein at a first vein location,
determine, based on the first plurality of characteristic vein diameters, whether the at least one vein has experienced a vein collapse during the first time period, and
determine a relation between IOP and ICP during the first time period, wherein if the at least one vein has experienced a vein collapse the IOP is determined to exceed the ICP.

In an example the system further comprises means for determining a heart pulse cycle of the person, and wherein said processing unit is further adapted to take into account temporal information about the heart pulse cycle, when determining the first plurality of characteristic vein diameters for the identified vein at a first vein location.

By taking into account temporal information about the heart pulse cycle, the determined first plurality of characteristic vein diameters may be correlated with the heart pulse cycle, which may help verifying the validity of the determined characteristic vein diameters, e.g. if a first characteristic vein diameter is measured during systole and a second characteristic vein diameter is measured shortly after in-between diastole and systole it may be expected to see a decrease in characteristic vein diameter.

The means for determining the heart pulse cycle may be an electrocardiograph machine or a heart rate sensor.

In an example the system further comprises means for determining a respiratory cycle of the patient, and wherein said processing unit is further adapted to take into account temporal information about the respiratory cycle and the recording of the image, when determining the relation between IOP and ICP during the first time period.

By taking account temporal information about the respiratory cycle, the determined first plurality of characteristic vein diameters may be correlated with the respiratory cycle, which may help verifying the validity of the determined characteristic vein diameters.

The means for determining the respiratory cycle may be an oximeter or a nasal cannula connected to a pressure transducer.

A feature described in relation to one of the aspects may also be incorporated in the other aspect, and the advantage of the feature is applicable to all aspects in which it is incorporated.

### BRIEF DESCRIPTION OF THE DRAWINGS

For exemplifying purposes, the disclosure will be described in closer detail in the following with reference to examples thereof illustrated in the attached drawings, wherein:

FIGS. 1a and 1b depict cross-sections of arteries and veins within an eye at different pressures and different relations between IOP and ICP.

FIG. 2 depicts a simplified pressure block model of a human body.

FIG. 3 depicts an example of a system according to the disclosure.

FIG. 4 depicts cross-sections of a vein and an associated artery during a cardiac cycle under two different relations between ICP and IOP.

FIG. 5 depicts a collapsed vein exiting the optic disc and two cross-sections of the vein at a first vein location and a second vein location.

FIG. 6 depicts a graph showing the characteristic vein diameter as a function of time and ICP.

FIGS. 7a and 7b depict graphs of measurements of AVR as a function of ICP.

FIG. 8 depicts a block diagram according to an example of the first aspect of the disclosure.

### DETAILED DESCRIPTION

In the following detailed description, examples of the present disclosure will be described. However, it is to be understood that features of the different examples are exchangeable between the examples and may be combined in different ways, unless anything else is specifically indicated. It may also be noted that, for the sake of clarity, the dimensions of certain elements illustrated in the drawings may differ from the corresponding dimensions in real-life implementations.

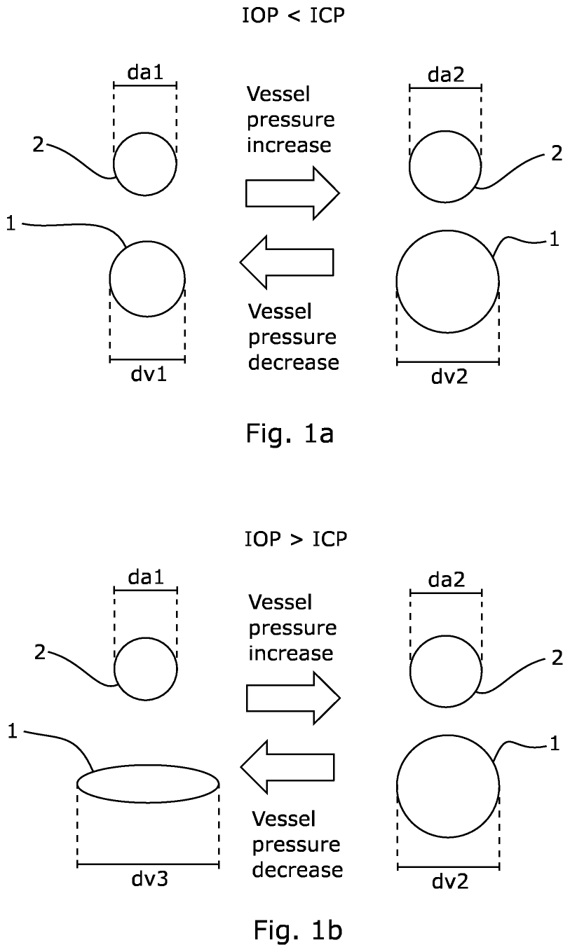

Referring initially to FIGS. 1a and 1b, depicting cross-sections of arteries 2 and veins 1 within an eye at different pressures and different relations between IOP and ICP. FIG. 1a depicts the situation where the ICP exceeds the IOP. The vein 1 has a first characteristic vein diameter dv1 and the artery 2 has a first characteristic artery diameter da1. As depicted on FIG. 1a the characteristic vein diameters dv1, dv2 normally exceeds the characteristic artery diameters da1, da2, because of the lower strength of the walls of the vein 1, though exceptions may occur. When the vessel pressure increases both the artery 2 and the vein 1 experiences a uniform increase in their cross-sections, consequently also an increase in their characteristic diameters.

In the context of the disclosure a vessel pressure is to be understood as the pressure inside of a blood vessel. The increase in the characteristic diameter leads to the artery 2 having a second characteristic artery diameter da2 and the vein 1 having a second characteristic vein diameter dv2. Furthermore, since the strength of the vein 1 is in general lower than that of the artery 2, the increase in the characteristic diameter is larger for the vein 1. When the vessel pressure decreases both the artery 2 and the vein 1 experiences a uniform decrease in their cross-sections, resulting in a decrease in their characteristic diameters. The decrease in the characteristic diameter leads to the artery 2 returning to the first characteristic artery diameter dal and the vein 1 returning to the first characteristic vein diameter dv1. FIG. 1b depicts the situation where the IOP exceeds the ICP. The general behaviour of the artery 2 does not change when the IOP exceeds the ICP. However, the behaviour of the vein 1 changes. When the vessel pressure decreases it results in the vein 1 collapsing. The collapse of the vein 1 leads to the vein 1 approaching an elliptical cross-section, wherein the characteristic vein diameter is measured along the semi major axis of the ellipse, resulting in the vein 1 having a third characteristic vein diameter dv3. The third characteristic diameter dv3 of the vein 1 in the collapsed state exceeds that of the first characteristic diameter dv1 and the second characteristic diameter dv2. Generally, the characteristic diameter of the vein in the collapsed state exceeds the characteristic diameter of the vein in the non-collapsed state.

Although cross-sections of the vein 1 and the artery 2 at different situations are shown, it is important to understand the change in the artery 2 and vein 1 happens continuously over time, thereby giving rise to a plurality of different cross-section for both the vein 1 and the artery 2.

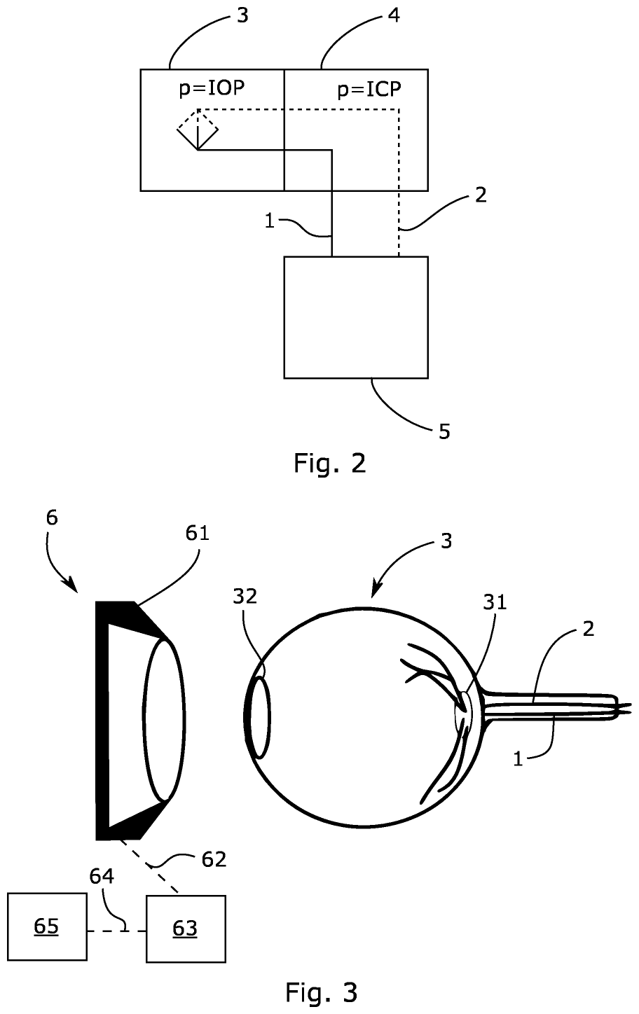

Referring to FIG. 2, which depicts a simplified pressure block model of a human body. To understand the nature of why a vein 1 collapses in the eye, it is highly relevant to understand the different pressures involved and how they interact. The depicted pressure block model has been simplified to only include a heart 5, a cranium 4, an eye 3, an artery 2, and a vein 1. The pressure within the veins 1 and arteries 2 are generated by the heart 5 pumping blood through the circulatory system. The pumping of the heart 5 generates a pressure wave, which drives the blood through the circulatory system and assures blood circulation within the vessels. The pressures in the blood vessels generated by the heart 5 causes the vessels to expand and contract as depicted in FIGS. 1a and 1b. To start of blood is pumped from the heart 5 to the cranium 4 through the artery 2. The artery 2 and vein 1 within the cranium 4 of a person experiences an external pressure exerting a pressure onto the vein 1 and the artery 2 in the cranium 4. The external pressure in the cranium 4 is known as ICP. From the cranium 4 the blood travels into the eye 3 via the artery 2. The artery 2 and the vein 1 within the eye 3 also experiences an external pressure. The external pressure in the eye 4 is known as IOP. ICP and IOP are rarely equal and in most cases differs from each other. When the blood is circulated back from the eye 3 it is done via the vein 1. The vein 1 travels from the eye 3 and back into the cranium 4 and then back to the heart 5. What is important to understand is that the blood vessels are not rigid tubes, but expand and contract, especially the vein 1 experience changes in size, as the strength of the vein is lower than that of the artery 2. Thus, it would be more accurate to view the blood vessels, in particular the vein 1, as a flexible tube capable of deforming. Thus, when either of the external pressures, IOP or ICP, pressing onto the vein 1 exceeds the pressure within the vein 1, the vein 1 collapses. Furthermore, for blood to flow from the eye 3 and into the cranium 4 through the vein 1, the pressure within the vein 1 must exceed the ICP otherwise a blood flow into the cranium 4 would not be present. The pressure generated by the pumping of the heart 5 ensures the blood pressure within the vein 1 exceeds the ICP and ensures a blood flow from the eye 3 and into the cranium 4. The pressure generated by the heart 5 may be described by the cardiac cycle. The pressure generated by the heart can be viewed as a pressure wave, which can roughly be split into systole where the blood pressure is high and diastole where the blood pressure is low. In a situation where the ICP exceeds the IOP, the vein 1 does not collapse since the blood pressure within the vein 1 will exceed the IOP, otherwise a proper blood flow would not be present. However, in the situation where the IOP exceeds the ICP, the blood pressure within the vein 1 may fall below the IOP causing a collapse of the vein 1, because of the external pressure, i.e. IOP, pressing down on the vein 1. However, the IOP may in some cases exceed the ICP without a vein collapse occurring, as the IOP needs to exceed the pressure within the vein in order for a vein collapse to occur. Consequently, if the pressure within the vein exceeds the IOP, the IOP may exceed ICP without a vein collapse occurring. Thus, only when the vein collapses may it be assured that the IOP exceeds the ICP. The collapse of the vein 1 will normally happen during diastole when the blood pressure is low. With the vein 1 returning from its collapse during systole. Of course, the higher the IOP is in relation to ICP, the larger is the amount of time the vein 1 will spend in a collapsed state during the cardiac cycle, as the blood pressure will fall below the IOP sooner during the pressure wave.

Referring to FIG. 3, which depicts an example of a system 6 according to the disclosure. The system 6 comprises an image recording device 61 connected to a processing unit 63. The image recording device 61 may be a smart phone provided with an add-on configured for ophthalmology measurements or a camera specifically configured for ophthalmology measurements. The connection 62 between the image recording device 61 and the processing device 63 may be a wired connection or a wireless connection. In some examples the image recording device 61 and the processing device may also be comprised in the same device, e.g. a smart phone, or other dedicated devices. The processing device 63 may further comprise a display 65 for displaying results determined by the processing unit 63. The display 65 may be connected to the processing unit 63 via a wired or wireless connection 64. The image recording device 61 is configured for recording images for of a retina part of an eye 3. Images are recorded through a lens 32 of the eye 3. The images recorded are meant for imaging veins 1 and arteries 2 on the retina part of the eye 3. In some examples the recorded images may also comprises images of an optic disc 31 of the eye 3. The characteristic diameters of veins and/or arteries determined from the recorded images is the diameter of the veins 1 and/or arteries 2 extending across the retina part of the eye 3. Consequently, when the veins 1 collapse they collapse against the retina part of the eye 3, thus resulting in an increase in the diameter of the veins 1 extending across the retina part of the eye 3, resulting in an increase in the characteristic vein diameter.

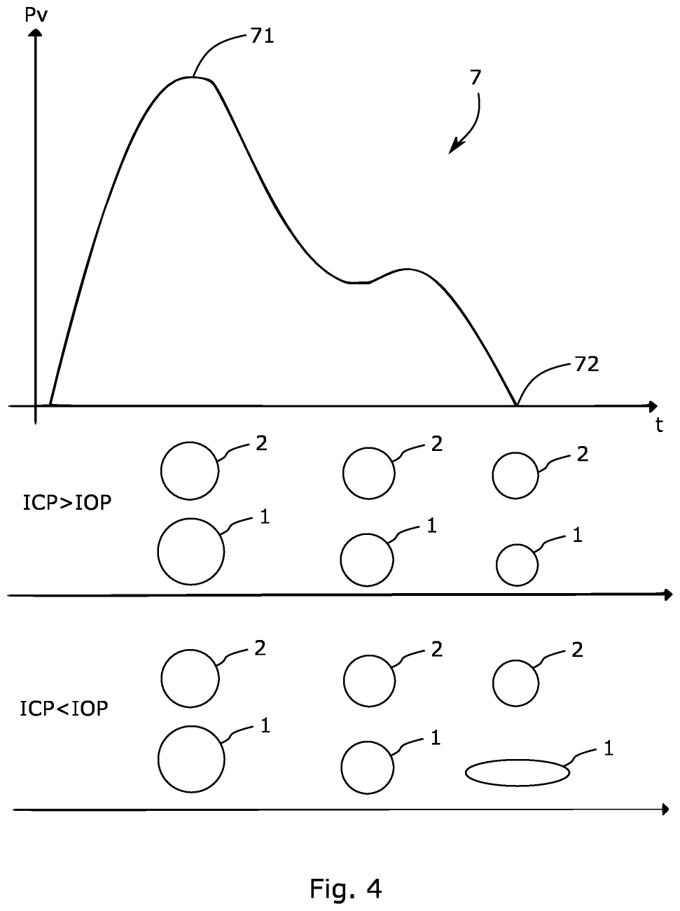

Referring to FIG. 4, which depict cross-sections of a vein 1 and an associated artery 2 during a cardiac cycle under two different relations between ICP and IOP. On FIG. 4 a graph 7 is depicted having time along a first axis and pressure within a vein 1 along a second axis. The graph 7 depicts the pressure within the vein 1 during the cardiac cycle. The pressure within the vein 1 shown on the graph 7 substantially follow the same trend as the arterial pressure during the cardiac cycle, other pressure behaviours within the vein 1 are also possible. During the cardiac cycle the pressure within the vein 1 changes significantly, the maximum pressure within the vein 1 happens at a systolic blood pressure 71. At the systolic blood pressure 71 the pressure within the artery 2 and the vein is at the maximum. In the situation where ICP exceeds IOP both the vein 1 and artery 2 have their maximum characteristic diameter at the systolic blood pressure 71. As the blood pressure decreases the characteristic diameters of both the artery 2 and the vein 1 decreases, until reaching their minimum characteristic diameter at a diastolic blood pressure 72. Thus, both the artery 2 and the vein 1 exhibits a matching behaviour when ICP exceeds IOP. In the situation where IOP exceeds ICP the behaviour of the artery 2 does not change. However, the behaviour of the vein 1 changes. As the blood pressure decreases towards the diastolic blood pressure 72, the vein 1 collapses as a result of the blood pressure within the vein 1 being exceeded by the IOP. The collapse of the vein 1 leads to an increased characteristic diameter at the diastolic blood pressure 72 which is opposed to what is observed for the artery 2, which will have a decreased characteristic diameter at the diastolic blood pressure 72. Thus, the artery 2 and the vein 1 exhibits differing behaviours. The characteristic diameter of the vein 1 resulting from the collapse of the vein 1, normally exceeds even the characteristic diameter of the vein 1 at the systolic blood pressure 71. Thus, by observing, determining and comparing the diameter behaviour of both the vein 1 and the artery 2, and subsequently comparing these to each other it is possible to determine, whether the vein 1 has undergone a collapse, since if the behaviours differs between the artery 2 and the vein 1, it signals that the vein 1 has undergone a collapse.

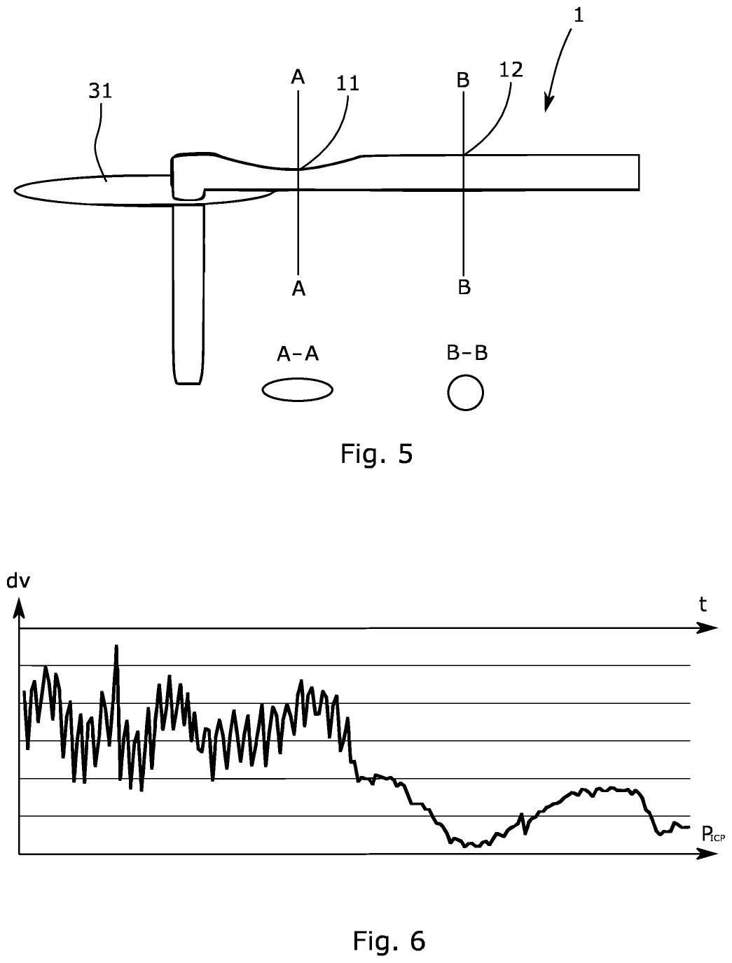

Referring to FIG. 5, which depicts a collapsed vein 1 exiting the optic disc 31 and two cross-sections of the vein 1 at a first vein location 11 and a second vein location 12. When the vein 1 collapses it has been observed to collapse close to the optic disc 31. The collapse of the vein 1 close to the optic disc 31 results in a pressure increase upstream of the collapse, which hinders the collapse from propagating to the rest of the vein 1. On FIG. 5 the vein has collapsed at a first vein location 11 close to the optic disc 31, the collapse results in the vein 1 having an elliptical cross-section A-A at the first vein location 11. Farther away from the optic disc 31, at the second vein location 12 no collapse has occurred as a result of the increased pressure caused by the collapse. Since no collapse occurs at the second vein location 12, the cross-section of the vein 1 at the second vein location is circular as seen from cross-section B-B. Since the cross-section B-B retains a circular cross-section, the characteristic diameter of the cross-section B-B will develop in accordance with the vessel pressure, i.e. whenever the vessel pressure decreases the characteristic diameter will decrease, and whenever the vessel pressure increases the characteristic diameter will increase. Whereas, at the cross-section A-A a decrease in vessel pressure may result in a collapse, which leads to an increase in the characteristic diameter. Thus, by observing, determining, and comparing the behaviour of the vein 1 at a first vein location 11 and a second vein location 12, it is possible to determine, whether the vein 1 has undergone a collapse, since if the behaviours differs between the first vein location 11 and the second vein location 12, it signals that the vein 1 has undergone a collapse.

Referring to FIG. 6, which depict a graph showing measurements of the characteristic vein diameter as a function of time and ICP. The measurements show that the characteristic vein diameter at low ICPs undergoes large changes over time. The large changes are caused by the vein collapsing. The vein collapsing results in a large change in the characteristic vein diameter, which corresponds to the large fluctuations seen on the graph. As the ICP increases the changes in the characteristic vein diameter becomes less pronounced, this is because the ICP starts to exceed the IOP, which leads to the vein not collapsing. However, small fluctuations are still present because of the changing blood pressure within the vein. The larger changes in characteristic vein diameter have been measured by the applicant to correspond to a relative change in the characteristic vein diameter above 2-3%, while the less pronounced changes correspond to a relative change in the characteristic vein diameter of 2-3% or below.

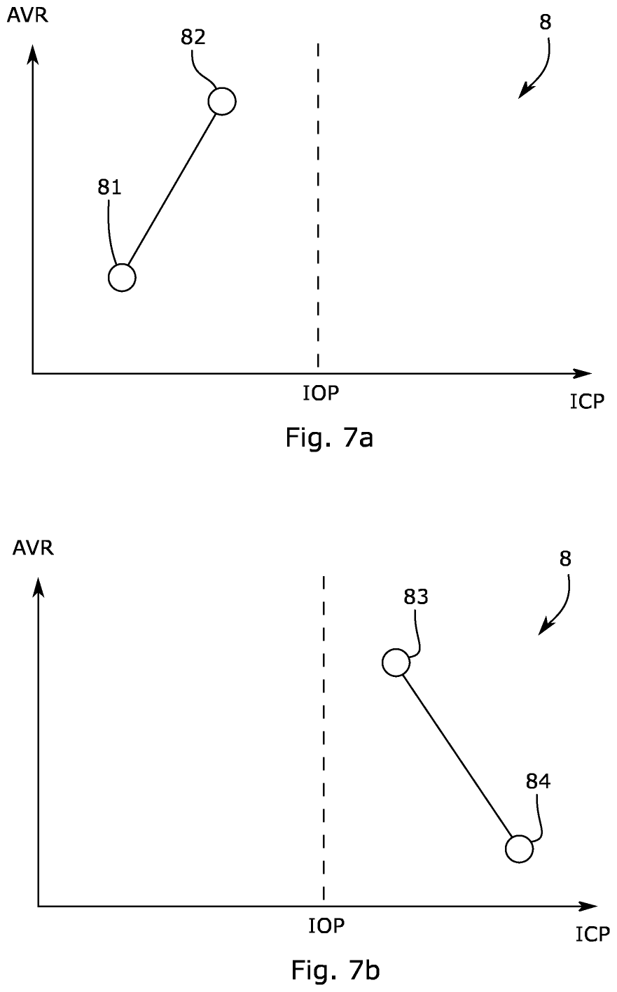

Referring to FIGS. 7a and 7b, which depict graphs 8 showing measurements of AVR as a function of ICP. On FIG. 7a two example measurements 81, 82 are shown of the AVR of a person. For both measurements 81, 82 the ICP was lower than the IOP. The first measurement 81 was performed for a first time period, and the second measurement 82 was performed for a second time period subsequent the first time period. Both measurements 81, 82 were carried out by recording a plurality of images and identifying one vein and at least one artery associated with said vein, then determining a first plurality of characteristic artery diameters for the identified artery at a first artery location and determining a first plurality of characteristic vein diameters for the identified vein at a first vein location. Based on the determined characteristic diameters of the vein and artery the AVR was calculated as a ratio between the determined characteristic diameters of the artery and the vein. The AVR may be calculated as a median or average AVR over the recorded time period. The AVR is normally a value between one and zero, as the vein generally exhibits a larger characteristic diameter than the artery. In the situation where ICP exceeds IOP, an increase in the AVR suggests a decrease in the ICP of a person. The reason why an increase in the AVR corresponds to a decrease in the ICP of a person, if ICP exceeds IOP, is that the vein deforms to a larger extent with pressure than the artery. Thus, when ICP falls the vein deforms to a smaller extent, thereby more closely resembling the artery and leading to an AVR close to one. However, the opposite is observed when IOP exceeds ICP, as is observed between the first measurement 81 and the second measurement 82, where the increase in the AVR corresponds to an increase in the ICP. In the situation where IOP exceeds ICP an increase in the AVR suggests an increase in the ICP. This behaviour is observed because of the vein collapsing. The collapse of the vein gives rise to a large change in characteristic diameter, thus giving a large difference between the vein and the artery. Consequently, the reason why the AVR increases between the first measurement 81 and the second measurement, even though the ICP increases, is that the time the vein is in the collapsed state during the cardiac cycle decreases as the ICP increases. Thus, the vein starts to resemble the artery more and more as the ICP starts to increase towards the IOP. Therefore, if a person is in a situation where IOP exceeds the ICP a false impression of the development of ICP may be developed if one is not aware of the relation between IOP and ICP.

On FIG. 7b another two example measurements 83, 84 are shown of the AVR of a person. The third measurement 83 was performed for a third time period, and the fourth measurement 84 was performed for a fourth time period subsequent the third time period. Both measurements 83, 84 were carried out in a similar manner as described in relation to the measurements 81, 82 of FIG. 7a. However, for both measurements the ICP exceeded the IOP. The result of the ICP exceeding the IOP is that the vein does not collapse. Therefore, the AVR decreases as the ICP increases, since the vein will start to deform more and more relative to the artery with increasing ICP. Thus, from FIGS. 7a and 7b the importance to know the relation between ICP and IOP is underlined, since it is needed to know the relation between IOP and ICP in order to correctly determine how the development in the AVR correlates to a development in the ICP.

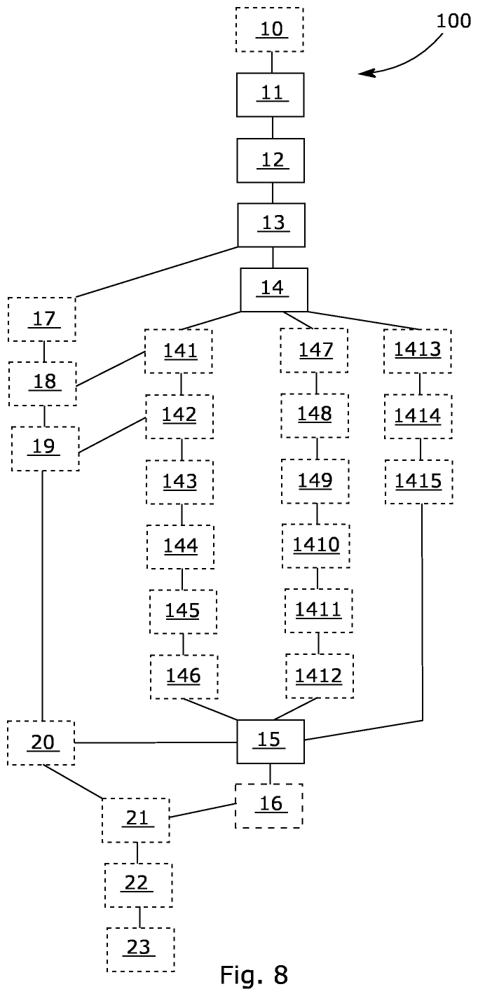

Referring to FIG. 8, which depicts a block diagram 100 according to an example of the first aspect of the disclosure. In a first step 10 a patient is prepared. The preparation of the patient may be voluntarily and is not a necessary step to carry out. In the preparation it may be desirable to dilate the pupil of an eye of the patient in order to record good quality images of a fundus of an eye of the patient. In a hospital environment there may be time and personnel for chemically dilating the pupil by e.g. dripping the patient's eye with belladonna. This could for instance be the case if the patient is known to suffer from specific conditions, such as hydrocephalus patients, patients with neurosurgical conditions, liver patients, kidney patients, or patients being observed for concussion. If, on the other hand, the patient is a victim of an accident and there is little time available, but an ambulance crew or paramedics suspect a head trauma, such as a developing hematoma, there may only be time for placing the patient in a dark environment, and e.g. ask him to look into the darkness over the shoulder of the person recording the image. The preparation of the patient may also comprise having the patient sitting/lying still for an amount of time in a recording position before initiating recording, which may allow a blood flow of the patient to normalize before starting recording. The amount of time may vary but is preferably 5-300 seconds.

In a second step 11 a plurality of images of the retina part of the eye of the person is recorded over a first time period. The recording of the images is carried out by using an image recording device. The recorded images should preferably be of the fundus of the eye with the optic disc in the middle of which the arteries and veins enter and exit the eye, respectively, along the optic nerve, and from which they branch out in all directions across the fundus. In principle, the recorded images may be recorded using any suitable device. This could be a dedicated device for this specific purpose. It could also be a digital camera with suitable optics, preferably in combination with a data processing device, such as a personal computer, PC, for inter alia processing the image data according to the method, and possibly providing storage capacity for the recorded images, at least temporarily. In particular, however, the image recording device could be the built-in camera of a smart phone fitted with a suitable lens adapter. The smart phone could thus be used both for the recording of the images, and the subsequent image data processing according to the method, as well as providing storage capacity for the recorded images. Suitable lens adapters for recording eye images are commercially available, such as the iExaminer™, from Welch Allyn, Inc., 4341 State Road, Skaneateles Falls, N.Y. 13153, USA. In general, but particular when using a smart phone as the image recording device, it should be noted that the image recording device should record the images in an uncompressed format, such as Bitmap (.bmp), Tagged Image File (.tiff), JPEG2000 in lossless setting (.JP2, .JPF, .JPX). Compression may blur images and therefore adversely affect the subsequent image data processing of the method according to the disclosure and is therefore not desirable.

In a third step 12 at least one vein is identified in the recorded plurality of images. The method is of course not limited to one vein, a plurality of veins may also be identified and analysed in a same manner as the at least one vein. The at least one vein may be identified manually by personnel analysing the plurality of recorded images. Alternatively, or in combination, the identification of the at least one vein may be carried out by a processing unit using appropriate image analysis software.

In a fourth step 13 a first plurality of characteristic vein diameters for the identified vein at a first vein location is determined, in a first plurality of images from the plurality of images recorded over the first time period.

In a fifth step 14 it is determined, based on the first plurality of characteristic vein diameters, whether the at least one vein has experienced a vein collapse during the first time period. The determination of whether the least one vein has experienced a vein collapse during the first time period may be carried out in plethora of ways. In the block diagram three different methods 141-146, 147-1412, 1413-1415 of determining whether the vein has collapsed in the first time period is presented. The different method may be carried all in parallel with each other, a combination of the methods may be chosen to be carried out in parallel, or just a single of the presented methods may be used.

In the first method 141-146 at least one artery associated with said vein is identified 141. The artery may be identified in a corresponding manner as the vein. A first plurality of characteristic artery diameters for the identified artery at a first artery location is determined 142 in the first plurality of images from the plurality of images recorded over the first time period. Based on the first plurality of characteristic artery diameters an artery diameter behaviour is determined 143. Based on the first plurality of characteristic vein diameters a vein diameter behaviour is determined 144. The vein diameter behaviour is compared 145 to the artery diameter behaviour. Based on the comparison between the vein diameter behaviour and the artery diameter behaviour, it is determined 146, whether the at least one vein has experienced a vein collapse during the first time period. The workings behind the first method 141-146 is presented in higher detail in relation to FIG. 4.

In the second method 147-1412 the location of the optic disc is determined 147 in the first plurality of images. A second plurality of characteristic vein diameters for the identified vein at a second vein location is determined 148 in the first plurality of images from the plurality of images recorded over the first time period. Based on the first plurality of characteristic vein diameters a first vein diameter behaviour is determined 149. Based on the second plurality of characteristic vein diameters a second vein diameter behaviour is determined 1410. The first vein diameter behaviour is compared 1411 to the second vein diameter behaviour. Based on the comparison between the first vein diameter behaviour and the second vein diameter behaviour, it is determined 1412, whether the at least one vein has experienced a vein collapse during the first time period. The workings behind the second method 147-1412 is presented in higher detail in relation to FIG. 5.

In the third method 1413-1415 a change in vein diameter during the first time period is determined 1413, based on the first plurality of characteristic vein diameters. The change in vein diameter is compared 1414 with a threshold value. Based on the comparison between the change in vein diameter and the threshold value it is determined 1415, whether the at least one vein has experienced a vein collapse during the first time period. The workings behind the third method 1413-1415 is presented in higher detail in relation to FIG. 5.

Thus, the determination made in the fifth step 14 rely on the first method 141-146, the second method 147-1412, the third method 1413-1415, or any combination of these, e.g. if it was not possible to identify an artery in the recorded images the second method 147-1412 and/or the third method 1413-1415 may still be used to determine whether the at least one vein has experienced a vein collapse. Alternatively, if an artery was identified but the image quality was only sufficient to measure a characteristic vein diameter at a first vein location, the first method 141-146 and/or the third method may be used. Being able to rely on several methods allows for verification of results and lowers the requirements on the recorded images.

In a sixth step 15 a relation between IOP and ICP during the first time period is determined. The determination is made based on whether the at least one vein has collapsed during the first time period, where if the at least one vein has experienced a vein collapse the IOP is determined to exceed the ICP.

In a non-mandatory seventh step 16 the previous steps 10-15 may be repeated for a second time period, allowing for the monitoring of a patient over a longer duration of time.

In parallel with determining the relation between IOP and ICP during the first time period, an AVR for the patient.

In an eight step 17 at least one artery associated with said vein is identified in said plurality of images. This step may in some case be skipped if the first method 141-146 is applied, as the first method involves identifying 141 at least one artery associated with the least one vein.

In a ninth step 18 a first plurality of characteristic artery diameters for the identified artery at a first artery location is determined in the first plurality of images from the plurality of images recorded over the first time period. Similarly, to the eight step 17, the ninth step 19 may also be skipped if the first method 141-146 is applied, as the first method involves determining 142 the first plurality of characteristic artery diameters for the identified artery at the first artery location.

In a tenth step 19 an AVR is calculated based on the first plurality of characteristic artery diameters and the first plurality of characteristic vein diameters. The calculated AVR may be calculated as a mean or median value based on the determined characteristic diameters.

In an eleventh step 20 the calculated AVR is compared to the relation between IOP and ICP, determined in the sixth step 15.

In a twelfth step 21 the eight step 17, ninth step 18, and tenth step 19 may be repeated for a second time period in order to determine an AVR for the second time period.

In a thirteenth step 22 the change in AVR between the first time period and the second time period is determined.

In a fourteenth step 23 the change in AVR is compared to the relation between IOP and ICP during the first time period and to the relation between IOP and ICP during the second time period.

Specific examples of the disclosure have now been described. However, several alternatives are possible, as would be apparent for someone skilled in the art. Such and other modifications must be considered to be within the scope of the present disclosure, as it is defined by the appended claims.

1. A method for a non-invasive assessment of a relation between an intracranial pressure and an intraocular pressure using an image recording device, comprising:
recording, over a first time period, a plurality of images of a retina part of an eye of a person using the image recording device,
identifying, in the plurality of images, at least one vein,
determining, in a first set of images from the plurality of images recorded over the first time period, a first plurality of characteristic vein diameters for the at least one vein at a first vein location,
determining, based on the first plurality of characteristic vein diameters, whether the at least one vein has experienced a vein collapse during the first time period, and
determining the relation between the intraocular pressure and the intracranial pressure during the first time period, wherein if the at least one vein has experienced a vein collapse the intraocular pressure is determined to exceed the intracranial pressure.

2. The method of claim 1, wherein determining whether the at least one vein has experienced a vein collapse comprises:
identifying, in the plurality of images, at least one artery associated with the at least one vein,
determining, in the first set of images from the plurality of images recorded over the first time period, a first plurality of characteristic artery diameters for the at least one artery at a first artery location,
determining, based on the first plurality of characteristic artery diameters, an artery diameter behaviour,
determining, based on the first plurality of characteristic vein diameters, a vein diameter behaviour,
comparing the vein diameter behaviour to the artery diameter behaviour, and
determining, based on the comparison between the vein diameter behaviour and the artery diameter behaviour, whether the at least one vein has experienced a vein collapse during the first time period.

3. The method of claim 1, wherein the plurality of images of the retina part of the eye are also of an optic disc of the eye, and wherein determining whether the at least one vein has experienced a vein collapse comprises:
determining, in the first set of images from the plurality of images recorded over the first time period, a location of the optic disc,
determining, in the first set of images from the plurality of images recorded over the first time period, a second plurality of characteristic vein diameters for the at least one vein at a second vein location, wherein the second vein location is farther away from the optic disc than the first vein location,
determining, based on the first plurality of characteristic vein diameters, a first vein diameter behaviour,
determining, based on the second plurality of characteristic vein diameters, a second vein diameter behaviour,
comparing the first vein diameter behaviour to the second vein diameter behaviour, and
determining based on the comparison between the first vein diameter behaviour and the second vein diameter behaviour, whether the at least one vein has experienced a vein collapse during the first time period.

4. The method of claim 3, wherein the second vein location is at least a distance corresponding to a diameter of the optic disc away from the optic disc.

5. The method of claim 1, wherein determining whether the at least one vein has experienced a vein collapse comprises:
determining, based on the first plurality of characteristic vein diameters, a change in vein diameter during the first time period,
comparing the change in the vein diameter with a threshold value, and
determining, based on the comparison between the change in the vein diameter and the threshold value, whether the at least one vein has experienced a vein collapse during the first time period.

6. The method of claim 1, further comprising:
recording, over a second time period, a second plurality of images of the retina part of the eye of the person using the image recording device,
identifying, in the second plurality of images, the at least one vein,
determining, in a second set of images from the second plurality of images recorded over the second time period, a second plurality of characteristic vein diameters for the at least one vein at the first vein location,
determining, based on the second plurality of characteristic vein diameters, whether the at least one vein has experienced a vein collapse during the second time period, and
determining the relation between the intraocular pressure and the intracranial pressure during the second time period, wherein if the at least one vein has experienced a vein collapse the intraocular pressure is determined to exceed the intracranial pressure.

7. The method of claim 1, further comprising:
identifying, in the plurality of images, at least one artery associated with the at least one vein,
determining, in the first set of images from the plurality of images recorded over the first time period, a first plurality of characteristic artery diameters for the at least one artery at a first artery location,
calculating an arteriovenous ratio based on the first plurality of characteristic artery diameters and the first plurality of characteristic vein diameters, and
comparing the arteriovenous ratio to the relation between the intraocular pressure and the intracranial pressure during the first time period.

8. The method of claim 6, further comprising:
identifying, in the second plurality of images, at least one artery associated with the at least one vein;
determining, in the second set of images from the plurality of images recorded over the second time period, a second plurality of characteristic artery diameters for the at least one artery at a first artery location,
calculating an arteriovenous ratio based on the second plurality of characteristic artery diameters and the second plurality of characteristic vein diameters,
determining a change in the arteriovenous ratio between the first time period and the second time period, and
comparing the change in the arteriovenous ratio to the relation between the intraocular pressure and the intracranial pressure during the first time period and to the relation between the intraocular pressure and the intracranial pressure during the second time period.

9. The method of claim 6, wherein the first time period, the second time period, or both is at least equal to a duration of at least one heart pulse cycle of the person, a duration of at least one respiratory cycle for the person, or both.

10. The method of claim 9, further comprising:
determining, based on the first plurality of characteristic vein diameters, the duration of at least one heart pulse cycle of the person, the duration of at least one respiratory cycle of the person, or both.

11. A system for performing a non-invasive assessment of a relation between an intracranial pressure and an intraocular pressure, comprising:
an image recording device, configured to record, over a first time period, a plurality of images of a retina part of an eye of a person, and
a processing unit communicatively connectable to the image recording device and configured to:
receive the plurality of images recorded by the image recording device,
identify, in the plurality of images, at least one vein,
determine, in a first set of images from the plurality of images recorded over the first time period, a first plurality of characteristic vein diameters for the at least one vein at a first vein location,
determine, based on the first plurality of characteristic vein diameters, whether the at least one vein has experienced a vein collapse during the first time period, and
determine the relation between the intraocular pressure and the intracranial pressure during the first time period, wherein if the at least one vein has experienced a vein collapse the intraocular pressure is determined to exceed the intracranial pressure.

12. The system of claim 11, further comprising:
a cardiac monitoring component configured to determine a heart pulse cycle of the person,
wherein the processing unit is further configured to determine the first plurality of characteristic vein diameters for the at least one vein at the first vein location based on temporal information about the heart pulse cycle.

13. The system of claim 11, further comprising:
a respiratory monitoring component configured to determine a respiratory cycle of the person,
wherein the processing unit is further configured to determine the first plurality of characteristic vein diameters for the at least one vein at the first vein location based on temporal information about the respiratory cycle.

14. The system of claim 11, wherein, to determine whether the at least one vein has experienced a vein collapse, the processing unit is further configured to:
identify, in the plurality of images, at least one artery associated with the at least one vein,
determine, in the first set of images from the plurality of images recorded over the first time period, a first plurality of characteristic artery diameters for the at least one artery at a first artery location,
determine, based on the first plurality of characteristic artery diameters, an artery diameter behaviour,
determine, based on the first plurality of characteristic vein diameters, a vein diameter behaviour,
compare the vein diameter behaviour to the artery diameter behaviour, and
determine, based on the comparison between the vein diameter behaviour and the artery diameter behaviour, whether the at least one vein has experienced a vein collapse during the first time period.

15. The system of claim 11, wherein, the plurality of images of the retina part of the eye are also of an optic disc of the eye, and wherein, to determine whether the at least one vein has experienced a vein collapse, the processing unit is further configured to:
determine, in the first set of images from the plurality of images recorded over the first time period, a location of the optic disc,
determine, in the first set of images from the plurality of images recorded over the first time period, a second plurality of characteristic vein diameters for the at least one vein at a second vein location, wherein the second vein location is farther away from the optic disc than the first vein location,
determine, based on the first plurality of characteristic vein diameters, a first vein diameter behaviour,
determine, based on the second plurality of characteristic vein diameters, a second vein diameter behaviour,
compare the first vein diameter behaviour to the second vein diameter behaviour, and
determine based on the comparison between the first vein diameter behaviour and the second vein diameter behaviour, whether the at least one vein has experienced a vein collapse during the first time period.

16. The system of claim 11, wherein, to determine whether the at least one vein has experienced a vein collapse, the processing unit is further configured to:
determine, based on the first plurality of characteristic vein diameters, a change in vein diameter during the first time period,
compare the change in the vein diameter with a threshold value, and
determine, based on the comparison between the change in the vein diameter and the threshold value, whether the at least one vein has experienced a vein collapse during the first time period.

17. The system of claim 11, wherein the processing unit is further configured to:
record, over a second time period, a second plurality of images of the retina part of the eye of the person using the image recording device,
identify, in the second plurality of images, the at least one vein,
determine, in a second set of images from the second plurality of images recorded over the second time period, a second plurality of characteristic vein diameters for the at least one vein at the first vein location,
determine, based on the second plurality of characteristic vein diameters, whether the at least one vein has experienced a vein collapse during the second time period, and
determine the relation between the intraocular pressure and the intracranial pressure during the second time period, wherein if the at least one vein has experienced a vein collapse the intraocular pressure is determined to exceed the intracranial pressure.

18. The system of claim 17, wherein the processing unit is further configured to:
identify, in the second plurality of images, at least one artery associated with the at least one vein;
determine, in the second set of images from the plurality of images recorded over the second time period, a second plurality of characteristic artery diameters for the at least one artery at a first artery location,
calculate an arteriovenous ratio based on the second plurality of characteristic artery diameters and the second plurality of characteristic vein diameters, determining a change in the arteriovenous ratio between the first time period and the second time period, and
compare the change in the arteriovenous ratio to the relation between the intraocular pressure and the intracranial pressure during the first time period and to the relation between the intraocular pressure and the intracranial pressure during the second time period.

19. The system of claim 11, wherein the processing unit is further configured to:
identify, in the plurality of images, at least one artery associated with the at least one vein,
determine, in the first set of images from the plurality of images recorded over the first time period, a first plurality of characteristic artery diameters for the at least one artery at a first artery location,
calculate an arteriovenous ratio based on the first plurality of characteristic artery diameters and the first plurality of characteristic vein diameters, and
compare the arteriovenous ratio to the relation between the intraocular pressure and the intracranial pressure during the first time period.

20. The system of claim 11, wherein the processing unit is further configured to:
determine, based on the first plurality of characteristic vein diameters, a duration of at least one heart pulse cycle of the person, a duration of at least one respiratory cycle of the person, or both.
